(19) United States
(12) Patent Application Publication
(10) Pub. No.: US 2023/0381437 A1
(43) Pub. Date: Nov. 30, 2023

(54) TRACHEOSTOMY INNER CANNULA WITH INTEGRATED FILTER

(71) Applicant: Covidien LP, Mansfield, MA (US)

(72) Inventors: Miles A. Curro, Boulder, CO (US); Stanley B. Kaus, Longmont, CO (US); Maurizio Listo, Modena (IT)

(73) Assignee: Covidien LP, Mansfield, MA (US)

(21) Appl. No.: 18/321,253

(22) Filed: May 22, 2023

Related U.S. Application Data
(60) Provisional application No. 63/346,482, filed on May 27, 2022.

(51) Int. Cl. A61M 16/04; A61F 2/01

(52) U.S. Cl. CPC A61M 16/0402; A61M 16/0465; A61F 2/01; A61F 2002/018

(57) ABSTRACT

Systems and methods for a tracheal tube inner cannula having an integrated filter are disclosed. In an example, the technology relates to a tracheal tube inner cannula that includes a proximal segment; a distal segment; a tapered segment attached to the proximal segment and the distal segment. The tapered segment includes a tapered lumen; and an integrated helical filter having a proximal filter diameter and a distal filter diameter, wherein the proximal filter diameter is greater than the distal filter diameter, the integrated helical filter having a ramped surface that wraps around a central axis at an angle such that the ramped surface extends distally through the integrated helical filter, the ramped surface including a coating to trap particulates as air flows through the integrated helical filter.

### CROSS-REFERENCE TO RELATED APPLICATIONS

This application claims the benefit of U.S. Provisional Application No. 63/346,482 filed May 27, 2022, entitled “Tracheostomy Inner Cannula With Integrated Filter,” which is incorporated herein by reference in its entirety.

### BACKGROUND

A tracheostomy tube is an artificial airway that passes through an opening (or stoma) in the neck and into the trachea to provide an alternative respiratory path for a patient. The tracheostomy procedure may be performed when a medical condition prevents normal breathing through the nose, mouth, or upper respiratory tract. A tracheostomy tube may be connected to a ventilator if required or can be left disconnected from the ventilator for spontaneously breathing patients. In cases where a patient breathes directly through a tracheostomy tube, without the support of a ventilator, the untreated air inhaled by the patient can pose risks. In normal breathing, inhaled air passes through the nose and mouth, which serve to warm and humidify the air, and provide some level of filtration of airborne foreign matter. A tracheostomy tube bypasses this mechanism and allows untreated air to pass directly to the delicate tissue of the lungs. Complications related to inhaling untreated air may include discomfort, increased coughing, infection, exposure to pathogens, and thickened respiratory mucus that can restrict or block the tracheostomy tube.

One tracheostomy tube design is a dual cannula arrangement. In this type of design, an outer cannula passes through the stoma and into the trachea, keeping the tracheostomy open. An inner cannula is inserted into the outer cannula and serves as the conduit for the passage of breathing gases. The inner cannula is secured within the outer cannula to prevent the inner cannula from being expelled during coughing or other activity. Different cannula designs use different mechanisms to secure the inner cannula. For example, pinch release and twist lock mechanisms are two commonly used approaches, among others. The ability to remove the inner cannula from the outer cannula is beneficial for maintaining the cleanliness and patency of the tracheostomy tube. For instance, most inner cannulas are disposable and are intended to be replaced on a daily basis. One example of a tracheostomy cannula system is described in U.S. Pat. No. 9,010,326, titled “Compressible Connector for an Inner Cannula,” which is incorporated herein by reference in its entirety.

It is with respect to this general technical environment that aspects of the present technology disclosed herein have been contemplated. Furthermore, although a general environment is discussed, it should be understood that the examples described herein should not be limited to the general environment identified herein.

### SUMMARY

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

In an aspect, the technology relates to a tracheal tube inner cannula that includes a proximal segment having a proximal opening; and proximal inner lumen having a first diameter. The inner cannula also includes a distal segment having a distal opening; and an inner distal lumen having a second diameter that is smaller than the first diameter. The inner cannula also includes a tapered segment attached to the proximal segment and the distal segment, the tapered segment includes a tapered lumen having a proximal diameter that is substantially the same as the first diameter and a distal diameter that is substantially the same as the second diameter; and an integrated filter having a proximal filter diameter and a distal filter diameter, wherein the proximal filter diameter is greater than the distal filter diameter and the second diameter.

In an example, the integrated filter is permanently affixed to a tube wall of the tapered lumen. In another example, the first diameter is at least 1.5 times greater than the second diameter. In yet another example, the integrated filter is a helical filter including a ramped filtering surface that wraps around a central axis. In a further example, the helical filter includes at least one spiral turn. In a still further example, the inner cannula further includes a membrane filter positioned distally from the helical filter. In another example, the inner cannula further includes a membrane filter positioned proximally from the helical filter. In still yet another example, the integrated filter further includes: a distal cylindrical portion that extends into the distal inner lumen, the distal cylindrical portion having a substantially constant diameter; and a frustoconical portion positioned proximally from the distal cylindrical portion.

In another aspect, the technology relates to a tracheal tube inner cannula that includes a proximal segment; a distal segment; a tapered segment attached to the proximal segment and the distal segment. The tapered segment includes a tapered lumen; and an integrated helical filter having a proximal filter diameter and a distal filter diameter, wherein the proximal filter diameter is greater than the distal filter diameter, the integrated helical filter having a ramped surface that wraps around a central axis at an angle such that the ramped surface extends distally through the integrated helical filter, the ramped surface including a coating to trap particulates as air flows through the integrated helical filter.

In an example, the proximal segment includes a proximal inner lumen having a first diameter; the distal segment includes an inner distal lumen having a second diameter that is smaller than the first diameter; and the tapered segment has a proximal diameter that is substantially the same as the first diameter and a distal diameter that is substantially the same as the second diameter. In another example, the inner cannula further includes a membrane filter positioned in the proximal segment. In a further example, the membrane filter is configured to filter a first size of particulates and the helical filter is configured to filter a second size of particulates, the first size being greater than the second size. In still another example, the inner cannula further includes a membrane filter positioned in the distal segment. In a further example, the membrane filter is configured to filter a first size of particulates and the helical filter is configured to filter a second size of particulates, the first size being less than the second size. In still yet another example, an outer conical surface of the helical filter contacts an interior surface of the tapered segment.

In another aspect, the technology relates to a tracheal tube assembly having an outer cannula and an inner cannula configured to be inserted into the outer cannula. The inner cannula includes a proximal segment having: a proximal opening; and a proximal inner lumen having a first diameter; a distal segment having: a distal opening; and an inner distal lumen having a second diameter that is smaller than the first diameter; a tapered segment attached to the proximal segment and the distal segment, the tapered segment having: a tapered lumen having a proximal diameter that is substantially the same as the first diameter and a distal diameter that is substantially the same as the second diameter; and an integrated helical filter positioned in at least one of the proximal segment, the distal segment, or the tapered segment, the integrated helical filter having a ramped surface that wraps around a central axis at an angle such that the ramped surface extends distally through the integrated helical filter.

In an example, the integrated helical filter is positioned in the tapered segment. In another example, the integrated helical filter has a diameter that is greater than the second diameter, and the integrated helical filter is positioned in at least one of the proximal segment or the distal segment. In yet another example, the integrated helical filter includes a frustoconical portion, positioned in the tapered segment, and a distal cylindrical portion positioned in the distal segment. In still another example, the ramped surface of the integrated helical filter includes a coating that traps particulates of air flowing through the integrated filter.

### BRIEF DESCRIPTION OF THE DRAWINGS

The following drawing figures, which form a part of this application, are illustrative of aspects of systems and methods described below and are not meant to limit the scope of the disclosure in any manner, which scope shall be based on the claims.

FIG. 1 depicts an example tracheal tube inner cannula with an integrated filter.

FIG. 2 depicts a cross-sectional view of the inner cannula.

FIG. 3 depicts an oblique view of the proximal portion of a single-turn filter.

FIG. 4 depicts an oblique view of the lateral and distal portions of the single-turn filter of FIG. 3.

FIG. 5 depicts an oblique view of the lateral and proximal portions of the single-turn filter of FIGS. 3-4.

FIG. 6 depicts a view of the proximal portion of the single-turn filter of FIGS. 3-5.

FIG. 7 depicts a lateral cross-sectional view of a single-turn filter.

FIG. 8 depicts a lateral view of a solid body representation of a single-turn filter.

FIG. 9 depicts a cross-sectional view of the proximal segment of an inner cannula, with another example of a filter.

FIG. 10 depicts a lateral view of the proximal segment of an inner cannula with an example filter having multiple spiral turns.

FIG. 11 depicts a lateral view of the proximal segment of an inner cannula with another example integrated filter.

FIG. 12 depicts a lateral view of the proximal segment of an inner cannula with a ribbon filtering element.

FIG. 13 depicts a cross-sectional view of the proximal segment of an inner cannula with a thin membrane filter positioned proximally from the integrated helical filter.

FIG. 14 depicts a cross-sectional view of the proximal segment of an inner cannula with a thin membrane filter placed distally from the integrated helical filter.

### DETAILED DESCRIPTION

A number of clinical scenarios exist in which a patient may receive respiratory intervention by way of a tracheostomy. In a tracheostomy, an opening (or stoma) is made through the neck and into the trachea. A tracheostomy tube is inserted into the stoma, bypassing the upper respiratory airway, and providing a direct path into the trachea for breathing. A small portion of the tracheostomy tube remains outside the body. This exterior portion may be connected to a medical ventilator for supplying oxygen or other gases to the patient, or the tracheostomy tube may be left unconnected from a ventilator, depending on the patient's health condition.

While a tracheostomy can be performed where short-term or acute clinical care is required, the procedure may be preferred in cases where prolonged ventilatory support is expected. In other cases, a tracheostomy may be performed where a patient is managing or being treated for an ongoing health condition in the upper respiratory tract. In such cases (or in other clinical scenarios) a patient may not require prolonged support from a ventilator, but it may still be preferable or medically necessary for the patient to rely on a tracheostomy tube for respiration. This type of use-case presents additional health concerns.

For example, because a tracheostomy tube bypasses the upper respiratory airway, the nose is no longer able to provide air filtration. Thus, the patient is at increased risk of inhaling airborne foreign matter, such as debris, allergens, or microorganisms, which can directly enter the delicate tissue of the lungs and trachea. In addition, the nose and mouth provide a mechanism for warming and humidifying the air entering the respiratory system. This mechanism helps maintain a level of moisture that promotes gas exchange within the lungs, and provides overall respiratory comfort. Maintaining adequate moisture also helps prevent a build-up of naturally occurring respiratory mucus. If this mucus becomes thick or excessive, it can be difficult to expel from the lungs, and may result in chronic coughing or lead to infection. Furthermore, excessive mucus can reduce or block airflow through the tracheostomy tube.

A patient whose tracheostomy tube is connected to a ventilator may not be nearly as prone to these clinical complications. Medical ventilators often supply clean, filtered oxygen or other gases to the patient, so the risk of exposure to airborne pollutants and pathogens is substantially reduced. Humidifiers used with ventilators are also capable of warming and humidifying the air delivered to a patient, which tends to diminish the build-up of mucus in the lungs and airways, and increase overall comfort.

However, to address the above complications in tracheostomy patients who are not reliant on a ventilator, other types of mitigations are beneficial. For instance, such patients are spontaneously breathing patients (e.g., breathing is controlled by their own efforts), and the air that is inhaled is that of the environment around them. The environmental air may include the types of airborne foreign matter discussed above, such as pollutants, debris, allergens, or microorganisms, among other types of particulates. As should be appreciated, inhaling such foreign matter is undesirable. In addition, the environmental air often has a temperature and humidification level that is less than optimal for the patient's lungs. One approach to help resolve this problem is to provide a removable and reusable Heat Moisture Exchanger (HME) that is connectable to an exterior portion of the tracheostomy tube. An HME is a type of passive humidifier that helps retain part of the warmth and moisture from a patient's exhalation and deliver the heat and moisture to the patient in the next inhalation. The HME also provides a level of filtration by trapping particulate matter in the material of the device as inhaled air passes through it. Alternatively, tracheostomy patients may prefer to use a cloth cover, such as a scarf, or a specifically designed tracheostomy bib to provide a similar function as an HME.

While current external HMEs have several benefits, there are also several drawbacks. One of the more widely cited concerns related to the use of externally connected HMEs is that patients experience a noticeable increase in airflow resistance as they breathe. For patients experiencing some degree of respiratory distress, or learning how to adapt to the tracheostomy tube, additional airflow limitations may not be well tolerated. In addition, HMEs add bulk to the exterior of the tracheostomy tube. This increased bulk may not be practical in all daily living situations, and some patients may be sensitive to the aesthetics of the device. Finally, HMEs require their own maintenance regimen separate from that of the tracheostomy tube, which itself requires regular, active maintenance, in order to prevent further health risks.

Similarly, patients who use a cloth cover or bib for humidification and filtration also encounter limitations during use. These types of mitigations may retain less of the warmth and moisture from each exhale than an HME. Because the cloth or bib is not tightly coupled to the tracheostomy tube, air filtration is less effective. Thus, while a cloth or bib may be more practical in some situations, and may be more aesthetically appealing, this type of mitigation may not be as efficacious as an HME.

The present technology addresses, among other things, the drawbacks and problems discussed above. More specifically, the present technology includes a tracheostomy tube inner cannula with an integrated filter that may be discarded with the inner cannula when a patient replaces the inner cannula, such as on a daily basis. The integrated filter provides a low resistance to airflow while still being able to filter contaminants from the air that is being inhaled by the patient. In addition to filtering, the integrated filter may also provide heat and moisture exchanging capabilities. Accordingly, the integration of the filter may eliminate the need for a bulky external HMEs, which also eliminates the need for additional cleaning or maintenance steps associated with these external HMEs. Furthermore, rather than using traditional filter material or thick membranes, which can restrict airflow to the patient, the present technology may utilize a helically shaped airflow path. This design produces a vortex of air inside the filter, which imparts centrifugal force on foreign matter traveling through the filter. The imparted force pushes airborne particles along the surface of a helical ramped surface and towards the peripheral surfaces of the filter. The helix and interior surfaces may be coated with a layer of a gel or adhesive that can trap particulate matter or neutralize airborne pathogens. The interior surfaces may also be porous or textured to trap contaminants within the structure of the filter. Because this filter design doesn't force air through a tightly packed filtration material or thick membrane, the resulting resistance to flow is substantially reduced.

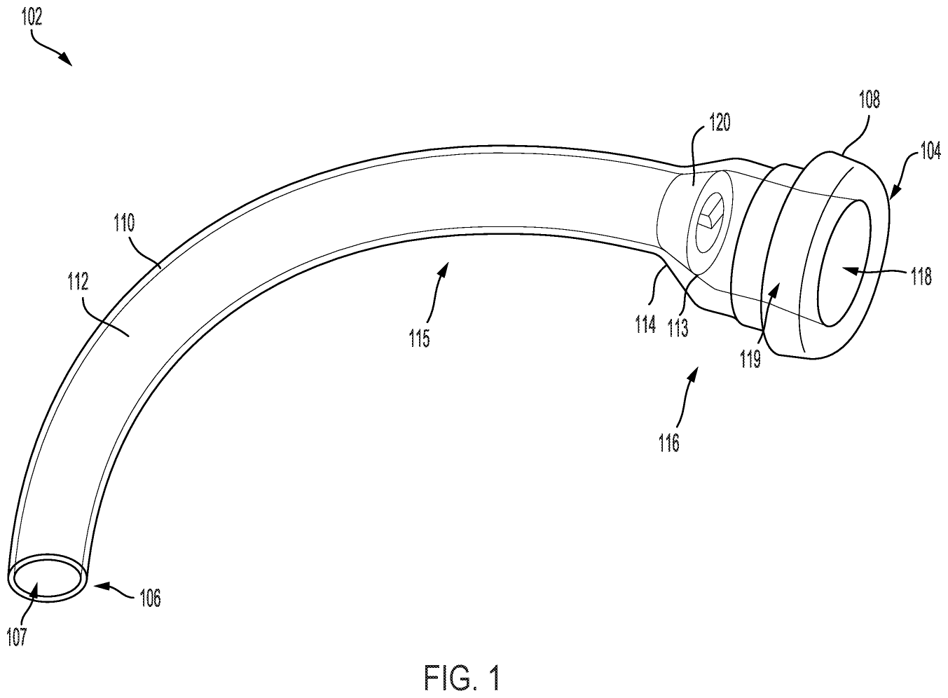

FIG. 1 depicts an example tracheal tube inner cannula 102 with an integrated filter 120. As depicted, the inner cannula 102 may be fabricated with an inherent bend, which facilitates insertion into a corresponding outer cannula of a tracheal tube assembly. The inner cannula 102 includes a proximal end 104 and a distal end 106. When the outer cannula of the tracheostomy tube is inserted within the patient, and the inner cannula 102 is installed, the distal end 106 is disposed inside the outer cannula and within the trachea of the patient. The proximal end 104 of the inner cannula 102 is disposed at the outer portion of the outer cannula, predominantly outside the body, or within or near the stoma.

The region or segment of the inner cannula 102 that is used to connect or secure the inner cannula 102 to an outer cannula may be referred to as a proximal segment 116 or a connection section. The proximal segment 116 is typically designed with a mechanism for securing the inner cannula 102 within the outer cannula. This mechanism helps prevent the inner cannula 102 from being expelled during coughing or other activity. Different cannula designs use different mechanisms to secure the inner cannula. For example, pinch release and twist lock mechanisms are two commonly used approaches, among others. However, the present technology is not limited to these specific examples. FIG. 1 depicts an inner cannula 102 that interfaces with the outer cannula by way of a flange 108. Various embodiments of the present technology may include a similar flange, multiple flanges, or no flange at all. Generally, the proximal segment 116 is designed as necessary to retain the inner cannula 102 within the outer cannula, and to either assist, or not interfere with the connection of external airway devices.

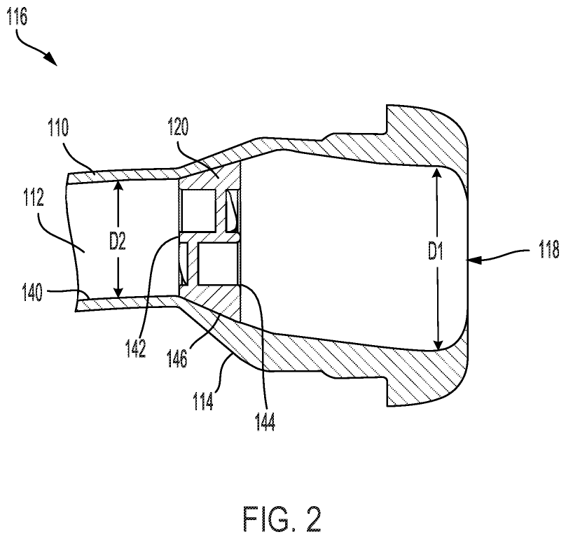

The proximal end 104 of the proximal segment 116 includes a proximal opening 118. Inhaled environmental air enters the inner cannula 102 via the proximal opening 118. A proximal inner lumen 119 of the proximal segment 116 is coupled to the proximal opening 118. The proximal inner lumen 119 has a first diameter D1 (FIG. 2) that may be the largest diameter of the lumen segments of the inner cannula 102.

The inner cannula 102 also includes a distal segment 115 that, in the example depicted, is made of a tube with tube wall 110 and an inner distal lumen 112 that has a substantially constant diameter. A distal opening 107 is positioned at the distal end 106 of the distal segment 115 to allow for air to flow into and out of the patient's lungs. The inner distal lumen 112 has a diameter D2 (FIG. 2) that is smaller than the diameter D1 of the proximal inner lumen 119. The diameter D1 may be 1.3 to 2.5 times greater than the diameter D2, among other possible sizes. The distal segment 115 is inserted within the patient, as discussed above.

The distal segment 115 and the proximal segment 116 are coupled together by a tapered segment 114. The tapered segment 114 starts at the diameter D1 of the proximal segment 116 and narrows to form the tube wall 110 of the distal segment 115. For instance, the tapered segment 114 is formed in a frustoconical shape where the outer diameter of the tapered segment 114, and the inner diameter of the tapered lumen 113 reduces in size from the proximal side to the distal side of the tapered segment 114. For instance, the proximal diameter of the tapered lumen 113 is substantially the same as the diameter D1 of the proximal segment, and the distal diameter of the tapered lumen 113 is substantially the same as the diameter D2 of the inner distal lumen 112.

An integrated filter 120 is disposed within the tapered segment 114 such that airflow between the distal inner lumen 112 and proximal opening 118 passes through the integrated filter 120. In the example depicted, the integrated filter 120 has a shape that matches the shape of the inner lumen of the tapered segment 114, as discussed further below.

FIG. 2 depicts a cross-sectional view of the inner cannula 102. In the example depicted, the integrated filter 120 is substantially frustoconical. For instance, the integrated filter 120 includes an outer conical surface 146 that abuts or contacts the inner cannula interior surface 140. The outer conical surface 146 is shaped or contoured to match the shape of the inner cannula interior surface 140 of the tapered segment 114. Accordingly, the outer conical surface 146 may be frustoconical where the interior surface 140 is frustoconical. Such a matching of shapes better secures the filter 120 in the tapered segment 114 and allows for a greater surface area for bonding the filter 120 to the interior surface 140.

The integrated filter 120 may affixed within the interior of the inner cannula 102 using various techniques. Examples may include, but are not limited to, medical grade adhesives, generalized bonding or molding techniques, or physical features or structures of the inner cannula interior that secure the integrated filter 120 in place. Furthermore, in some examples, the integrated filter 120 itself may be designed such that its shape, or other physical features or structures of it, may facilitate fixation within the inner cannula 102.

As discussed above, the integrated filter 120 depicted in FIG. 2 is substantially frustoconical, where a distal filter diameter of the distal filter surface 142 is less than that of a proximal filter diameter of the proximal filter surface 144. Additionally, the proximal filter diameter of the proximal filter surface 144 is larger than the diameter of the distal inner lumen 112. This arrangement prevents the integrated filter 120 from being dislodged and inhaled, since no orientation of the integrated filter 120 would permit it to physically pass through the smaller inner distal lumen 112. Such a safety measure may be implemented through a wide variety of shapes, sizes, or embodiments, and is not limited to a filter with a frustoconical shape. The diameter of the distal filter surface 142, however, may be the same as or less than the diameter of the distal inner lumen 112.

The cross-sectional representation depicted in FIG. 2 also illustrates portions of the interior of the integrated filter 120. The example filter 120 is a spiral or helix design that causes a vortex of air to be created when air flows through the filter 120. Rather than using traditional filter material or thick membranes, which can restrict airflow to the patient, the present technology may utilize a helically shaped filter that with a spiral or helical airflow path. This design produces a vortex of inhaled air inside the filter, which imparts centrifugal force on foreign matter traveling through the filter. The imparted force pushes airborne particles along the surface of the helix and towards the peripheral surfaces on the interior of the filter.

The helix and interior surfaces may be coated with a layer of a gel or adhesive that can trap particulate matter and/or neutralize airborne pathogens. The interior surfaces may also be porous or textured to trap contaminants within the structure of the filter. Because this helical filter 120 does not force air through a tightly packed filtration material or thick membrane, the resulting drop in air pressure is substantially reduced, which provides less resistance to breathing. The example filter 120 depicted in FIG. 2 includes a single spiral. The spiral shape and different variations thereof are depicted in further detail below.

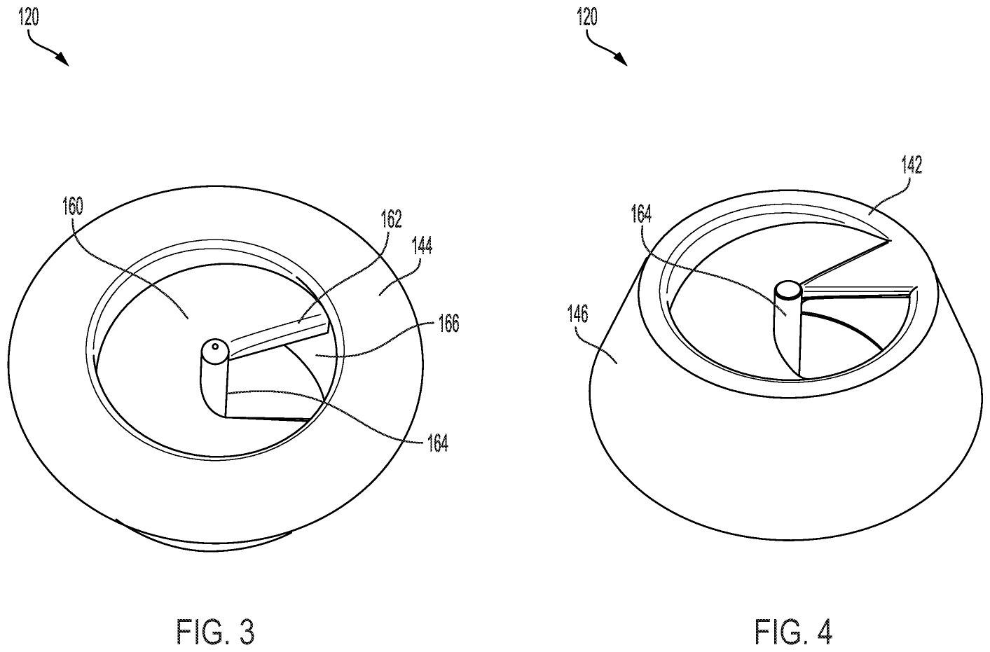

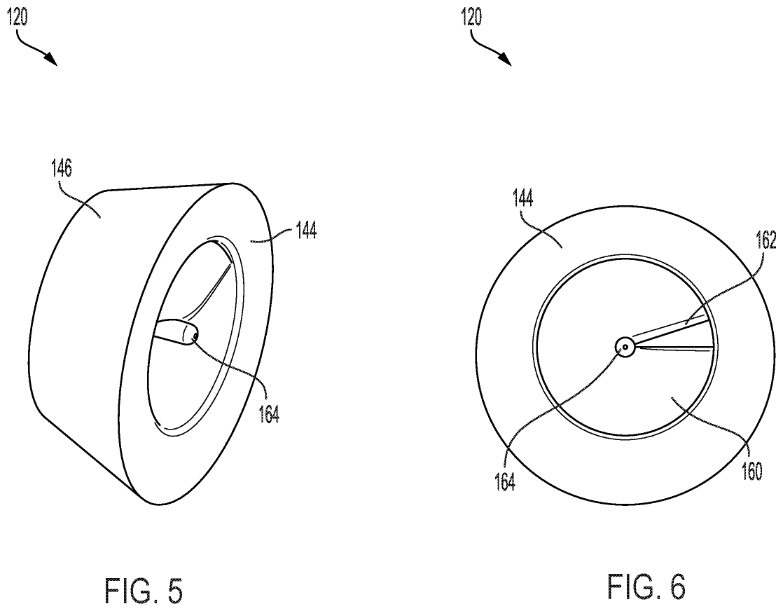

FIG. 3 depicts an oblique view of a proximal portion of a single-turn filter 120. FIG. 4 depicts an oblique view of the lateral and distal portions of the single-turn filter 120. FIG. 5 depicts an oblique view of the lateral and proximal portions of the single-turn filter 120. FIG. 6 depicts a view of the proximal portion of the single-turn filter 120. FIGS. 3-6 are discussed concurrently.

The helical filter 120 includes a ramped filtering surface 160 that wraps around a center hub 164 in a spiral or helical manner. The ramped filtering surface is also ramped at an angle from a proximal side of the filter 120 to a distal side of the filter 120. During inhalation, air deflects along a filtering surface 160 as the air travels down and around the center hub 164 towards the distal end 106 of the inner cannula 102. To pass through the filter 120, the airflow is directed in a circular motion resulting from this interaction with the angled or ramped filtering surface 160. The circular motion imparts centrifugal force on any airborne foreign material that may be present in the inhaled air, which forces these contaminants towards the interior surface walls 166 of the integrated filter 120. The proximal filter surface 144, filtering surface 160, leading edge 162 of the spiral, center hub 164, and interior surface walls 166 may all be pretreated or coated with a gel, adhesive, or other coating, which may trap the contaminants and/or neutralize any airborne pathogens. Additionally or alternatively, the interior surfaces may also be porous or textured to trap contaminants of a specific size, thereby removing them from the inhaled air.

The continued opening of the spiral results in minimal airflow restriction compared to externally connected HMEs. In addition, the coatings and/or other material used to form the filter 120 may also retain heat and moisture of the air exhaled by the patient. In such examples, the filter 120 may function as an integrated or internal HME that may be referred to as an integrated HME filter 120. When the exhaled air interacts with the underside of the spiral-shaped filtering surface 160, the exhaled air travels through the filter in the opposite direction as the inhaled air, and the exhaled air undergoes similar forces and vortex motion as the inhaled air.

The outer conical surface 146 can be more easily seen in FIG. 4. As discussed above, the conical surface 146 may be shaped and sized to match the interior surface 140 of tapered segment 114 of the inner cannula 102. In such examples, the interior surface 140 effectively provides a seat for the conical surface 146 of the filter 120. Positioning of the filter 120 into the inner cannula 102 during manufacturing is also improved due to the matching conical shapes, which aids in alignment of the filter 120 inside the inner cannula 102.

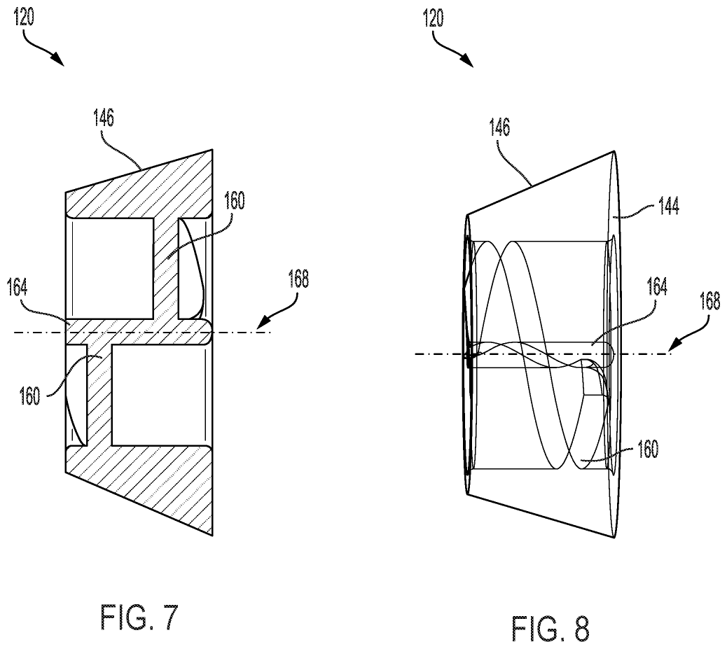

FIG. 7 depicts a cross-sectional view of the integrated filter 120. FIG. 8 depicts the integrated filter 120 from a similar vantage point as FIG. 7, but the view in FIG. 8 is a solid-body representation, rather than cross-sectional view. FIGS. 7-8 are discussed concurrently. The substrate material that forms the bulk of the conical portions of the filter is continuous with the filtering surface 160 and center hub 164. In other embodiments, the filtering surface 160 and center hub 164 may be made from different material than the non-filtering, conical portions of the integrated filter 120. Although these surfaces may be embodied in different substrate materials, there remains a structural continuity between these features. The central hub 164 may extend along a central axis 168 of the integrated filter 120. The central axis 168 extends through the center of the inner lumen in a proximal to distal direction. In some examples, the center hub 164 may also be omitted and the ramped surface 160 may be connected to, or protrude from, the wall of the inner lumen.

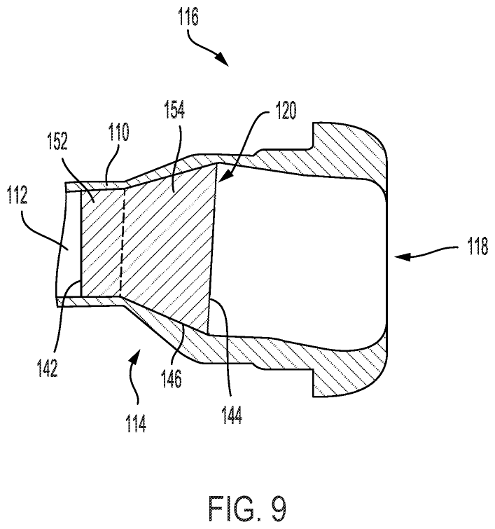

FIG. 9 depicts a cross-sectional view of the proximal segment 116 of an inner cannula 102 with another example filter 120. Similar to the filters discussed above, at the distal end of the tapered segment 114, the frustoconical portion 154 of the filter has a predominantly cylindrical shape that is larger than the diameter of the distal inner lumen 112. The filter 120 depicted in FIG. 9, however, has a different configuration at the distal end of the filter 120. At the distal end of the filter 120, a portion of the filter 120 extends into the distal inner lumen 112. The portion extending to the inner distal lumen 112 is a distal cylindrical portion 152.

This distal cylindrical portion 152 of the filter 120 may have a constant diameter as compared to the frustoconical portion 154 that has a changing diameter from the proximal end to the distal end. The diameter of the cylindrical portion 152 may be substantially the same as the diameter D2 of the inner distal lumen 112. For instance, the diameter of the cylindrical portion 152 may be at least 98% of the diameter D2 of the inner distal lumen 112. As such the outer surface of the cylindrical portion 152 may contact the interior surface of the distal inner lumen 112 and may be bonded or otherwise joined to the interior surface of the distal inner lumen 112.

By incorporating the distal cylindrical portion 152 in addition to the frustoconical portion 154, the integrated filter 120 can be made larger to achieve a desired level of air filtration or to affect heat and moisture retention. For instance, the overall length of the filter 120 (measured in a distal to proximal direction) may be made larger, which can allow for additional spiral turns in a helical example of the integrated filter 120. The additional spiral turns may provide for additional filtration and/or heat and moisture retention, but the additional spiral turns also add additional resistance to airflow. As such the number of spiral turns may be configured depending on the environment type and/or the patient type. For example, for highly polluted environments, the filter 120 may be configured with additional turns. In contrast, for low-pollution environments, the filter 120 may be configured with fewer turns to reduce the air resistance of the filter 120.

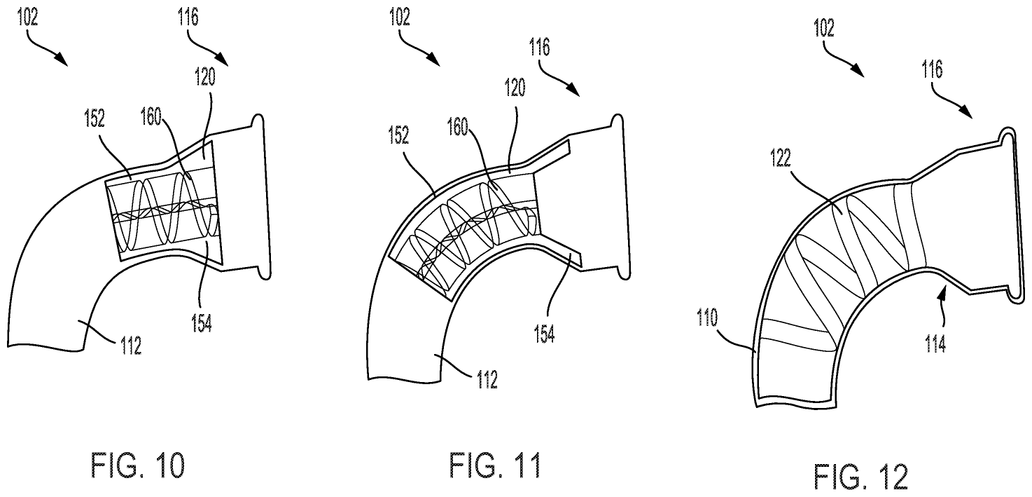

FIG. 10 depicts a lateral view of the proximal segment 116 of an inner cannula 102 with an example filter 120 having multiple spiral turns. The number of spiral turns may be defined as the number of revolutions that the ramped filtering surface 160 makes around the center hub 164. For instance, a single turn may be defined as the ramped filtering surface 160 making one full revolution around the center hub 164. The filter 120 depicted in FIG. 10 has a similar configuration as the filter 120 depicted in FIG. 9. For instance, the filter 120 in FIG. 10 has a frustoconical portion 154 and a distal cylindrical portion 152. In the example depicted in FIG. 10, the ramped filtering surface 160 extends through both the frustoconical portion 154 and a distal cylindrical portion 152. Despite the outer diameter of the frustoconical portion 154 changing, the width of the spiral-shaped filtering surface 160 may remain constant across the frustoconical portion 154 and the distal cylindrical portion 152. In other examples, the width of the spiral-shaped filtering surface 160 may change proportionally with the diameter size of the outer conical surface 146.

In the example depicted in FIG. 10, the additional spiral turns are achieved by extending the filter 120 further into the distal inner lumen 112. Alternatively or additionally, the filter 120 may be extended towards and into the proximal segment 116. For example, the filter 120 may have a frustoconical portion 154 ad a proximal cylindrical portion extending from the proximal side of the frustoconical segment 116.

FIG. 11 depicts a lateral view of the proximal segment 116 of an inner cannula 102 with another example integrated filter 120. In this example, the bulk of the filter substrate has been shifted out of the frustoconical portion 154 and into the inner distal lumen 112. For instance, the filter 120 still includes a distal cylindrical portion 152 and a frustoconical portion 154. However, the frustoconical portion 154 is substantially devoid of filtration material. For instance, the ramped surface 160 may begin at the start of the inner distal lumen 112. In some examples, the ramped surface 160 may also extend partially into the frustoconical portion 154, but the ramped surface does not extend into more than 20% or 10% of the length of the frustoconical portion 154 (measured from the distal side to the proximal side of the frustoconical portion 154). Instead of providing a filtering function, the frustoconical portion 154 primarily serves as a securing function the filter 120. For instance, the frustoconical portion 154 may be substantially hollow to allow air to pass. The outer edges of the frustoconical portion 154 (e.g., the outer conical surface 146) may still be bonded or otherwise attached to the interior surface 140 of the tapered segment 114. The outer diameter of the frustoconical portion 154 at the proximal end is also still greater than the diameter D2 of the inner distal lumen 112. Accordingly, the frustoconical portion 154 prevents the filter 120 from passing through the inner distal lumen 112.

In examples where the frustoconical portion 154 does not provide filtering functionality, the distal cylindrical portion 152 may be extended to allow for more spiral turns of the ramped surface 160. For instance, the length of the cylindrical portion 152 (measured from a distal to a proximal side) may be greater than the length of the frustoconical portion 154. As an example, the length of the distal cylindrical portion 152 may be at least 1.5 times the length of the frustoconical portion 154. In contrast, in examples where the frustoconical portion 154 also provides filtering functionality, the distal cylindrical portion 152 may have a length that is less than the length of the frustoconical portion 154. For instance, the length of the distal cylindrical portion 152 may be 0.5 times the length of the frustoconical portion 154. In yet other example, the frustoconical portion 154 may perform the filtering functions and include the ramped surface 160, while the distal cylindrical portion 152 may be substantially devoid of the ramped surface. In such examples, the distal cylindrical portion 152 serves to position and secure the filter 120 in the example inner cannula 102.

In examples where the distal cylindrical portion 152 extends into the inner distal lumen 112, the distal cylindrical portion 152 may be curved to match the inherent bend of the inner distal lumen 112, which may assist insertion of the inner cannula 102 into the outer cannula. In examples where the substrate material of the integrated filter 120 is compliant, no preset curve in the filter 120 may be required as the distal cylindrical portion 152 bends with the inner cannula 102 as the filter 120 is inserted.

FIG. 12 depicts a lateral view of the proximal segment 116 of an inner cannula 102 with a ribbon filtering element 122 or ribbon 122. The ribbon filtering element 122 may include a band of adhesive or other material that can collect contaminants or foreign matter. The band of adhesive is bonded to the interior walls of the inner lumen in a spiral manner. As foreign matter passes by the ribbon 122, the foreign matter may attach to the ribbon 122 instead of flowing into the lungs of the patient. While not shown in FIG. 12, the ribbon 122 may be combined with a helical filter 120 that causes the inhaled air to swirl and force the foreign matter the exterior surfaces of the lumen 112. The force generated by the swirling air more effectively causes the foreign matter to be collected by the ribbon 122. The ribbon 122 may extend down a substantial portion of the inner distal lumen 112. For example, the ribbon 122 may extend across more than 20%, 50%, or 80% of the length of the inner distal lumen 112.

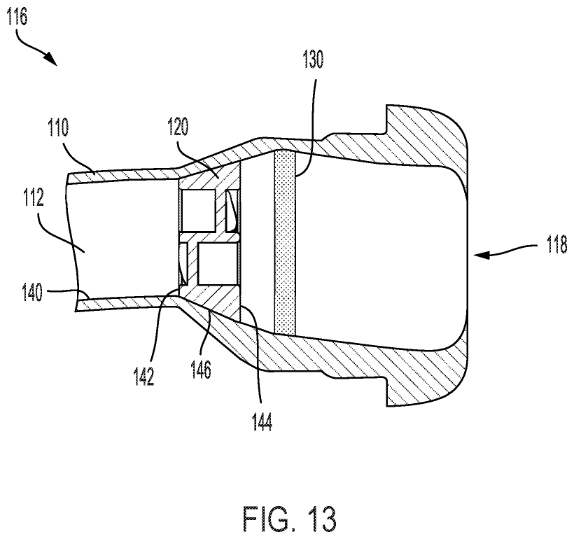

FIG. 13 depicts a cross-sectional view of the proximal segment 116 of an inner cannula 102, a helical filter 120, and a filter membrane 130. The thin filter membrane 130 is provided in addition to the helical filter 120 to provide further filtration of the inhaled air. The filter membrane 130 may be made relatively thin to also help reduce pressure drops across the filter membrane, which can provide resistance to breathing efforts. The filter membrane 130, however, may still provide more airflow resistance than the helical filter 120. Because the filter membrane 130 is not the sole filter, the filter membrane 130 may be made thinner and thus provide less resistance to airflow than a normal filter membrane.

The filter membrane 130 and the helical filter 120 may be configured for different filtering purposes. For instance, the filter membrane 130 may be selected to remove specific sizes, types, or species of contaminants or foreign matter, leaving the remaining foreign matter to be removed by the helical filter 120. As an example, the filter membrane 130 may be configured to remove larger particles than the helical filter 120. Such a configuration may be appropriate where the filter membrane 130 is placed proximally to the helical filter 120 (e.g., the filter membrane is positioned between the helical filter 120 and the proximal opening 118).

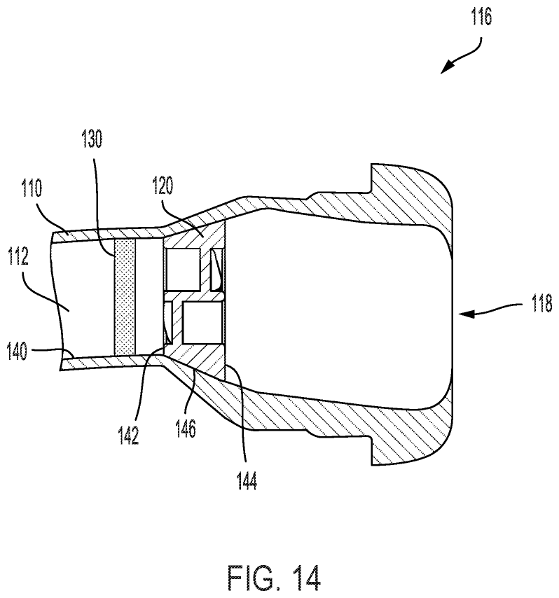

FIG. 14 depicts a cross-sectional view of the proximal segment 116 of an inner cannula with the thin membrane filter 130 placed distally from the integrated helical filter 120. Unlike the example depicted in FIG. 13, the example in FIG. 14 has the thin membrane filter placed distally from the helical filter 120. The filter membrane 130 may be located in the inner distal lumen 112, the tapered segment 114, or in the proximal segment 116. In such a configuration, the filter membrane still serves to filter foreign matter from the inhaled air and the filter membrane 130 may still be configured to filter different foreign matter than the helical filter 120. For instance, the helical filter 120 may be configured to filter larger particulates than the filter membrane 130.

While the inner cannula 102 described herein is intended to be paired with an appropriate outer cannula, the inner cannula 102 may be used in any respiratory application where it can be suitably accommodated. The combination of inner and outer cannulas forms a tracheostomy tube that is capable of being used in conjunction with other external airway devices. Examples include, but are not limited to, temperature and humidity control equipment, sensors, gas analyzers, other active or passive measurement devices, passive tubing, tubing interconnects or adapters, ventilators, etc. While an additional filter may not typically be used when a patient is connected to a ventilator, examples of the present technology may be suitable for use with a ventilator or other pressure generating device—especially where the gases being delivered to the patient may not be filtered (or may not be adequately filtered) by the ventilator. The integrated filter 120 may also be used to complement the filtration performed by the ventilator or ventilator circuit. For instance, the filtration performed by the ventilator may be configured for a particular particulate size or type and the integrated filter 120 may be configured for a different size or type.

Those skilled in the art will recognize that the methods and systems of the present disclosure may be implemented in many manners and as such are not to be limited by the foregoing aspects and examples. In other words, functional elements may be performed by a single or multiple components. In this regard, any number of the features of the different aspects described herein may be combined into single or multiple aspects, and alternate aspects having fewer than or more than all of the features herein described are possible. Functionality may also be, in whole or in part, distributed among multiple components, in manners now known or to become known.

Further, as used herein and in the claims, the phrase “at least one of element A, element B, or element C” is intended to convey any of: element A, element B, element C, elements A and B, elements A and C, elements B and C, and elements A, B, and C. In addition, one having skill in the art will understand the degree to which terms such as “about” or “substantially” convey in light of the measurement techniques utilized herein. To the extent such terms may not be clearly defined or understood by one having skill in the art, the term “about” shall mean plus or minus ten percent.

Numerous other changes may be made which will readily suggest themselves to those skilled in the art and which are encompassed in the spirit of the disclosure and as defined in the appended claims. While various aspects have been described for purposes of this disclosure, various changes and modifications may be made which are well within the scope of the disclosure. Numerous other changes may be made which will readily suggest themselves to those skilled in the art and which are encompassed in the spirit of the disclosure and as defined in the claims.

What is claimed is:

1. A tracheal tube inner cannula comprising:
a proximal segment comprising:
a proximal opening; and
a proximal inner lumen having a first diameter;
a distal segment comprising:
a distal opening; and
an inner distal lumen having a second diameter that is smaller than the first diameter; and
a tapered segment attached to the proximal segment and the distal segment, the tapered segment comprising:
a tapered lumen having a proximal diameter that is substantially the same as the first diameter and a distal diameter that is substantially the same as the second diameter; and
an integrated filter having a proximal filter diameter and a distal filter diameter, wherein the proximal filter diameter is greater than the distal filter diameter and the second diameter.

2. The tracheal tube inner cannula of claim 1, wherein the integrated filter is permanently affixed to a tube wall of the tapered lumen.

3. The tracheal tube inner cannula of claim 1, wherein the first diameter is at least 1.5 times greater than the second diameter.

4. The tracheal tube inner cannula of claim 1, wherein the integrated filter is a helical filter including a ramped filtering surface that wraps around a central axis.

5. The tracheal tube inner cannula of claim 4, wherein the helical filter includes at least one spiral turn.

6. The tracheal tube inner cannula of claim 4, further comprising a membrane filter positioned distally from the helical filter.

7. The tracheal tube inner cannula of claim 4, further comprising a membrane filter positioned proximally from the helical filter.

8. The tracheal tube inner cannula of claim 1, wherein the integrated filter further comprises:
a distal cylindrical portion that extends into the distal inner lumen, the distal cylindrical portion having a substantially constant diameter; and
a frustoconical portion positioned proximally from the distal cylindrical portion.

9. A tracheal tube inner cannula comprising:
a proximal segment;
a distal segment;
a tapered segment attached to the proximal segment and the distal segment, the tapered segment comprising:
a tapered lumen; and
an integrated helical filter having a proximal filter diameter and a distal filter diameter, wherein the proximal filter diameter is greater than the distal filter diameter, the integrated helical filter having a ramped surface that wraps around a central axis at an angle such that the ramped surface extends distally through the integrated helical filter, the ramped surface including a coating to trap particulates as air flows through the integrated helical filter.

10. The tracheal tube inner cannula of claim 9, wherein:
the proximal segment includes a proximal inner lumen having a first diameter;
the distal segment includes an inner distal lumen having a second diameter that is smaller than the first diameter; and
the tapered segment has a proximal diameter that is substantially the same as the first diameter and a distal diameter that is substantially the same as the second diameter.

11. The tracheal tube inner cannula of claim 9, further comprising a membrane filter positioned in the proximal segment.

12. The tracheal tube inner cannula of claim 11, wherein the membrane filter is configured to filter a first size of particulates and the helical filter is configured to filter a second size of particulates, the first size being greater than the second size.

13. The tracheal tube inner cannula of claim 9, further comprising a membrane filter positioned in the distal segment.

14. The tracheal tube inner cannula of claim 13, wherein the membrane filter is configured to filter a first size of particulates and the helical filter is configured to filter a second size of particulates, the first size being less than the second size.

15. The tracheal tube inner cannula of claim 9, wherein an outer conical surface of the helical filter contacts an interior surface of the tapered segment.

16. A tracheal tube assembly having an outer cannula and an inner cannula configured to be inserted into the outer cannula, the inner cannula comprising:
a proximal segment comprising:
a proximal opening; and
a proximal inner lumen having a first diameter;
a distal segment comprising:
a distal opening; and
an inner distal lumen having a second diameter that is smaller than the first diameter;
a tapered segment attached to the proximal segment and the distal segment, the tapered segment comprising:
a tapered lumen having a proximal diameter that is substantially the same as the first diameter and a distal diameter that is substantially the same as the second diameter; and
an integrated helical filter positioned in at least one of the proximal segment, the distal segment, or the tapered segment, the integrated helical filter having a ramped surface that wraps around a central axis at an angle such that the ramped surface extends distally through the integrated helical filter.

17. The tracheal tube assembly of claim 16, wherein the integrated helical filter is positioned in the tapered segment.

18. The tracheal tube assembly of claim 16, wherein the integrated helical filter has a diameter that is greater than the second diameter, and the integrated helical filter is positioned in at least one of the proximal segment or the distal segment.

19. The tracheal tube assembly of claim 16, wherein the integrated helical filter includes a frustoconical portion, positioned in the tapered segment, and a distal cylindrical portion positioned in the distal segment.

20. The tracheal tube assembly of claim 16, wherein the ramped surface of the integrated helical filter includes a coating that traps particulates of air flowing through the integrated helical filter.
